(19) United States
(12) Patent Application Publication
(10) Pub. No.: US 2024/0350001 A1
(43) Pub. Date: Oct. 24, 2024

(54) Systems and Methods for Predicting Corneal Improvement From Scheimpflug Imaging Using Machine Learning

(71) Applicant: Mayo Foundation for Medical Education and Research, Rochester, MN (US)

(72) Inventors: Sanjay V. Patel, Rochester, MN (US); Jon J. Camp, Rochester, MN (US); David O. Hodge, Jacksonville, FL (US); David R. Holmes, III, Rochester, MN (US)

(21) Appl. No.: 18/685,847

(22) PCT Filed: Aug. 23, 2022
(86) PCT No.: PCT/US2022/041230

§ 371 Date: Feb. 22, 2024

Related U.S. Application Data
(60) Provisional application No. 63/236,069, filed on Aug. 23, 2021.

(51) Int. Cl. A61B 3/10; G06V 10/764; G16H 20/00; G16H 30/40

(52) U.S. Cl. CPC A61B 3/1005; G06V 10/764; G16H 20/00; G16H 30/40

(57) ABSTRACT

Corneal improvement following a therapeutic procedure, such as Descemet membrane endothelial keratoplasty (“DMEK”), is predicted or otherwise monitored using Scheimpflug imaging and a machine learning model that has been trained to predict or otherwise monitor corneal improvement from Scheimpflug imaging parameters that are independent of corneal thickness. The machine learning model can include a predictive model trained using ensemble learning, or other suitable machine learning models such as neural networks.

### BACKGROUND

Fuchs endothelial corneal dystrophy (“FECD”) is a common cause of corneal transplantation and currently the subject of novel therapeutic interventions that will require clinical trials. In FECD, edema forms in the cornea causing it to swell and thicken. Treatment of FECD is necessary when edema is visible on clinical examination; however, clinically-significant edema is frequently not visible by clinical examination (e.g., subclinical edema), making the decision to proceed to treatment more difficult. The standard treatment for FECD is Descemet membrane endothelial keratoplasty (“DMEK”), a type of corneal transplantation. Since DMEK is an invasive procedure that can have complications, there is a need for predicting whether and/or when a patient would be a good candidate for the procedure.

FECD encompasses a wide range of severity based on the functional state of the corneal endothelium. When corneal edema is clinically-detectable, patients usually have vision symptoms. and in advanced cases may also have pain from bullae. Treatment of FECD is indicated when edema is clinically-detectable, and DMEK usually results in a reduction of central corneal thickness (“CCT”) with improvement in vision. When clinically-detectable corneal edema is not visible, patients can still be symptomatic because of the presence of subclinical edema. Subclinical edema can be detected by assessing for three specific patterns in Scheimpflug tomography posterior elevation and pachymetry maps, and treatment by DMEK can also result in significant improvement of corneal function and vision, and reduction in CCT.

As the medical and surgical treatment landscape for FECD continues to evolve, developing an objective method of measuring and predicting improvement in corneal function will be important for clinical trial outcomes and for application in clinical practice. Measurements of CCT have previously been used as a guideline for considering keratoplasty in clinical practice; however, clinical decisions based on absolute values of CCT can result in inappropriate treatment (a change in CCT over time is more helpful than isolated values of CCT). Scheimpflug tomography has the potential to objectively quantify corneal edema and its improvement with therapy. A model for predicting edema resolution after DMEK was recently proposed by D. Zander, et al., in “Predicting Edema Resolution after Descemet Membrane Endothelial Keratoplasty for Fuchs Dystrophy Using Scheimpflug Tomography,” JAMA Ophthalmol., 2021; 139(4):423-430; however, this model was largely dependent on preoperative CCT and therefore subject to the same caveats of using CCT measurements in clinical practice. Predicting the presence of corneal edema in FECD from CCT alone is not possible and, therefore, models that are strongly dependent on preoperative CCT are limited in their accuracy.

### SUMMARY OF THE DISCLOSURE

The present disclosure addresses the aforementioned drawbacks by providing a method for predicting corneal improvement using Scheimpflug imaging. The method includes accessing Scheimpflug imaging data with a computer system, where the Scheimpflug imaging data have been acquired from a subject using a Scheimpflug imaging system. A predictive model is also accessed with the computer system. The predictive model has been constructed to predict corneal improvement following a therapy based on preoperative Scheimpflug imaging data. As one example, ensemble learning can be used to learn the parameters for use in the predictive model. The Scheimpflug imaging data are applied to the predictive model, generating output as corneal improvement feature data that indicate a predicted corneal improvement following the therapy. The corneal improvement feature data can then be presented to a user.

It is another aspect of the present disclosure to provide a method for predicting corneal improvement using Scheimpflug imaging. The method includes accessing Scheimpflug imaging data with a computer system, where the Scheimpflug imaging data have been acquired from a subject using a Scheimpflug imaging system and include Scheimpflug imaging parameters that are independent of corneal thickness. A trained machine learning model is also accessed with the computer system. The machine learning model has been trained to predict corneal improvement following a therapy based on preoperative Scheimpflug imaging data. The Scheimpflug imaging data are applied to the trained machine learning model, generating output as corneal improvement feature data that indicate a predicted corneal improvement following the therapy. The corneal improvement feature data can then be presented to a user.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a flowchart setting forth the steps of an example method for predicting corneal improvement using one or more predictive models that have been trained to predict corneal improvement following a therapy based on preoperative Scheimpflug imaging.

FIG. 2 is a flowchart setting forth the steps of an example method for training a predictive model to predict corneal improvement based on Scheimpflug imaging.

FIG. 3 illustrates an example of predicting corneal improvement from Scheimpflug imaging in a first patient and/or subject using the systems and methods described in the present disclosure.

FIG. 4 illustrates an example of predicting corneal improvement from Scheimpflug imaging in a second patient and/or subject using the systems and methods described in the present disclosure.

FIG. 5 is a block diagram of an example system for predicting corneal improvement based on Scheimpflug imaging.

FIG. 6 is a block diagram of example components that can implement the system of FIG. 5.

### DETAILED DESCRIPTION

Described here are systems and methods for predicting or otherwise monitoring corneal improvement following a therapeutic procedure, such as Descemet membrane endothelial keratoplasty (“DMEK”). In general, the systems and methods described in the present disclosure use Scheimpflug imaging and a model (e.g., a specialized computer analysis or a machine learning model) that has been trained to predict or otherwise monitor corneal improvement from Scheimpflug imaging parameters that are independent of corneal thickness. The systems and methods described in the present disclosure have the ability to predict improvement based on pre-intervention Scheimpflug images, and therefore can be used to assess disease progression and regression.

In general, a predictive model can be trained on Scheimpflug images, Scheimpflug tomography maps, and/or parameters computed, derived, or generated from such images and/or maps. In some instances, one or more predictive models can be trained using ensemble learning techniques, such as boosting techniques. As a non-limiting example, gradient boosting can be used to learn the predictive model.

As a non-limiting example, Scheimpflug pachymetry patterns can be more advantageous for predicting corneal improvement than pachymetry values. Thus, it is an aspect of the systems and methods described in the present disclosure to quantify Scheimpflug pachymetry patterns to provide an objective assessment of corneal edema and its improvement after therapy. For instance, Scheimpflug tomography can detect subclinical edema in FECD and predict disease prognosis based on the presence of specific posterior elevation and pachymetry map patterns. As such, in some embodiments, a model is constructed or otherwise derived from an analysis of Scheimpflug images that yields parameters measuring tomography map patterns.

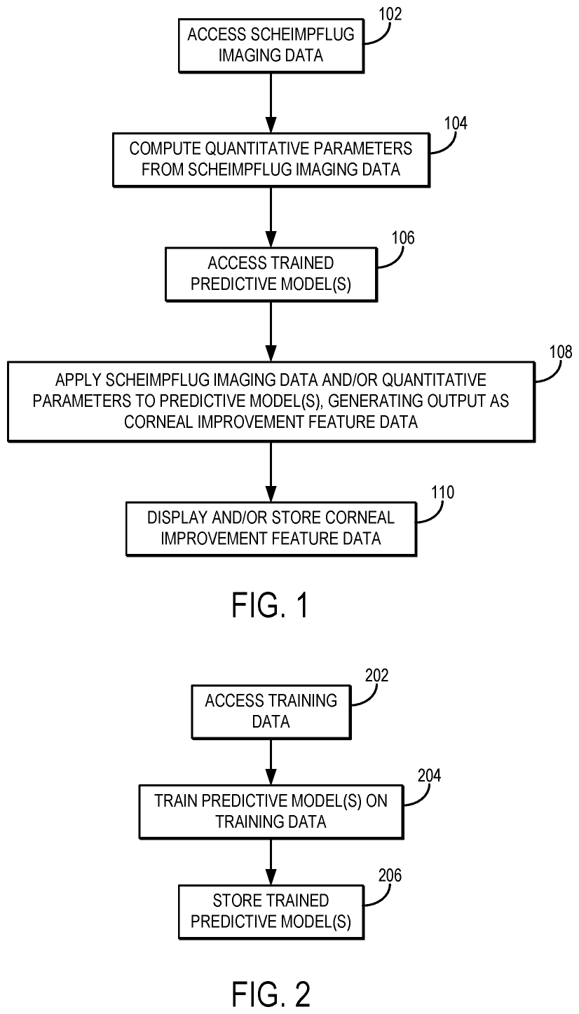

Referring now to FIG. 1, a flowchart is illustrated as setting forth the steps of an example method for predicting or otherwise monitoring corneal improvement following a therapeutic procedure, such as DMEK, using a predictive model or other machine learning model.

The method includes accessing Scheimpflug imaging data with a computer system, as indicated at step 102. Accessing the Scheimpflug imaging data may include retrieving such data from a memory or other suitable data storage device or medium. Alternatively, accessing the Scheimpflug imaging data may include acquiring such data with a Scheimpflug imaging system and transferring or otherwise communicating the data to the computer system, which may be a part of the Scheimpflug imaging system.

The Scheimpflug imaging data may include Scheimpflug images acquired with a Scheimpflug imaging system and/or a three-dimensional (“3D”) tomographic reconstruction generated from such images. Additionally or alternatively, the Scheimpflug imaging data may include tomographic maps computed, derived, or otherwise generated from Scheimpflug images. For example, the Scheimpflug imaging data may include tomographic maps such as curvature maps, elevation maps, and/or pachymetry maps. As a non-limiting example, the Scheimpflug imaging data may include posterior elevation and pachymetry maps.

In still other implementations, quantitative parameters can be computed, derived, or otherwise generated from Scheimpflug images and/or tomographic maps, as indicated at step 104. Alternatively, these quantitative parameter data may be accessed as part of the Scheimpflug imaging data.

In some implementations, the quantitative parameters can be computed, derived, or otherwise generated from tomographic maps such as posterior elevation and pachymetry maps. As a non-limiting example, the quantitative parameters may include irregular isopachs, displacement of the thinnest point of the cornea, and/or volume of posterior depression. As another non-limiting example, quantitative parameters may include isopach regularity (e.g., circularity and eccentricity) in the pachymetry map(s), the radius of the posterior corneal surface, and/or the mean and standard deviation of corneal thickness at different diameters from the center.

As a non-limiting example, the Scheimpflug imaging data (e.g., posterior elevation and pachymetry maps) can be analyzed—whether automatically, semi-automatically, or manually—to provide quantitative parameters related to parameters associated with, corresponding to, or otherwise relevant with respect to subclinical edema. As noted, example parameters include irregular isopachs, displacement of the thinnest point of the cornea, and volume of posterior depression. Additionally or alternatively, other quantitative parameters can also be computed from the Scheimpflug imaging data. As noted above, in some instances the quantitative parameters may include instrument derived parameters (i.e., parameters that are exported from the Scheimpflug imaging system's software) as potential factors for predicting postoperative improvement after therapy. As noted, quantitative parameters derived from the Scheimpflug imaging data can include patterns of subclinical edema, such as measures of isopach regularity, displacement of the thinnest point from the pupil center, and volume of posterior tissue depression. Instrument-derived parameters can also be related to the posterior elevation and pachymetry maps, such as radius and asphericity of the posterior surface, and mean and standard deviation of corneal thickness at different diameters from the center.

A trained, or otherwise constructed, predictive model, or other suitable machine learning model, is then accessed with the computer system, as indicated at step 106. Accessing the predictive model may include accessing model parameters (e.g., predictive input parameters, model coefficients, weights, biases, or combinations thereof) that have been optimized or otherwise estimated by training the predictive model on training data. In some instances, retrieving the predictive model can also include retrieving, constructing, or otherwise accessing the particular predictive model structure to be implemented. For instance, data pertaining to the predictive model (e.g., number of predictive parameters to input, type of predictive parameters to input) may be retrieved, selected, constructed, or otherwise accessed.

In general, the predictive model is trained, or has been trained, on training data in order to predict corneal improvement following a therapy, such as DMEK. As a non-limiting example, the predictive model is trained on Scheimpflug imaging data and/or parameters computed from such imaging data, in order to predict improvement following therapies. In some embodiments, the predictive model is constructed based on model parameters that are learned through a training process, such as using ensemble learning. For example, the predictive model can be constructed using regression (e.g., linear regression) based on a combination of predictive parameters (e.g., quantitative parameters estimated or otherwise derived from Scheimpflug imaging data), where the predictive parameters may be identified using ensemble learning.

The Scheimpflug imaging data are then input to the predictive model(s), generating output as data indicating a prediction of corneal improvement, as indicated at step 108. For example, the output data may include corneal improvement feature data, which that indicate a quantitative or probabilistic measure of corneal improvement. As one example, the output data may include values of predicting change in central corneal thickness, ΔCCT. As another example, the output data may include maps depicting the predicted spatial distribution of change in corneal thickness over a region of the cornea. As still another example, the output data may include an indication of predicted corneal improvement, which may include a classification, quantitative score, probability of improvement, or other parameters associated with or indicating predicted corneal improvement following a therapy, such as DMEK.

The corneal improvement feature data generated by inputting the Scheimpflug imaging data to the trained predictive model(s) can then be displayed to a user, stored for later use or further processing, or both, as indicated at step 110. As an example, the corneal improvement feature data may be displayed to a user by displaying a value (e.g., a predicted change in central corneal thickness, a quantitative score of predicted improvement) or image (e.g., a map of predicted corneal thickness) that indicates the predicted corneal improvement.

Referring now to FIG. 2, a flowchart is illustrated as setting forth the steps of an example method for training one or more predictive models (e.g., machine learning or other statistical models) on training data, such that the one or more predictive models are trained to receive input as Scheimpflug imaging data in order to generate output as corneal improvement feature data, which that indicate a quantitative or probabilistic measure of corneal improvement.

In general, the predictive model(s) can be generated using any number of suitable model construction techniques, including using boosting algorithms that convert weak learners into one or more strong learners. For instance, the predictive model(s) can be produced using adaptive boosting (“AdaBoost”), gradient boosting, extreme gradient boosting (“XGBoost”), or the like.

Alternatively, the predictive model(s) could be constructed using other techniques (e.g., other ensemble learning techniques, such as bootstrap aggregating, or “bagging”), or could be replaced with other suitable machine learning algorithms or models, such as those based on supervised learning, unsupervised learning, deep learning, ensemble learning, reinforcement learning, and so on. In some instances, the predictive model(s) can include one or more neural networks (e.g., convolutional neural networks) that have been trained to generate corneal improvement feature data that that indicate a quantitative or probabilistic measure of corneal improvement based on patterns in Scheimpflug imaging data, including tomographic maps computed from Scheimpflug images.

The method includes accessing training data with a computer system, as indicated at step 202. Accessing the training data may include retrieving such data from a memory or other suitable data storage device or medium. Alternatively, accessing the training data may include acquiring such data with a Scheimpflug imaging system and transferring or otherwise communicating the data to the computer system, which may be a part of the Scheimpflug imaging system.

In general, the training data can include Scheimpflug images, Scheimpflug tomography maps (e.g., curvature maps, elevation maps, pachymetry maps), and/or quantitative parameters computed or otherwise derived from such images and/or maps. The training data can be collected from groups of subjects, and can include preoperative data, postoperative data, or both.

Additionally or alternatively, the method can include assembling training data from Scheimpflug imaging data using a computer system. This step may include assembling the Scheimpflug imaging data into an appropriate data structure on which the predictive model(s) can be trained. Assembling the training data may include assembling Scheimpflug imaging data, segmented Scheimpflug imaging data, and other relevant data. For instance, assembling the training data may include generating labeled data and including the labeled data in the training data. Labeled data may include Scheimpflug imaging data, segmented Scheimpflug imaging data, or other relevant data that have been labeled as belonging to, or otherwise being associated with, one or more different classifications or categories. The labeled data may include labeling all data within a field-of-view of the Scheimpflug imaging data and/or segmented Scheimpflug imaging data, or may include labeling only those data in one or more regions-of-interest within the Scheimpflug imaging data and/or segmented Scheimpflug imaging data. The labeled data may include data that are classified on a voxel-by-voxel basis, or a regional or larger volume basis.

Additionally or alternatively, assembling the training data may include implementing one or more data augmentation processes. As one example data augmentation process, cloned data can be generated from the Scheimpflug imaging data. As an example, the cloned data can be generated by making copies of the Scheimpflug imaging data while altering or modifying each copy of the Scheimpflug imaging data. For instance, cloned data can be generated using data augmentation techniques, such as adding noise to the original Scheimpflug imaging data, performing a deformable transformation (e.g., translation, rotation, both) on the original Scheimpflug imaging data, smoothing the original Scheimpflug imaging data, applying a random geometric perturbation to the original Scheimpflug imaging data, combinations thereof, and so on.

One or more predictive models (or other suitable machine learning models) are trained on the training data, as indicated at step 204. In general, the predictive model(s) can be trained using ensemble learning techniques. As one non-limiting example, ensemble learning techniques such as bagging and/or boosting may be used. For instance, the predictive model(s) may be trained using a boosting technique, such as adaptive boosting, gradient boosting, extreme gradient boosting, or the like.

In general, boosting techniques allocate weights to each weak learner model during the training stage. Using gradient boosting, as an example, the predictive model determines the relative influence of each parameter for predicting improvement. The parameters with the highest relative influences can be identified as predictive input parameters. In some non-limiting examples, preoperative CCT can be excluded as an input parameter for the model.

In a non-limiting example, the relative influence of all possible parameters predictive of the improvement in CCT were summarized, and the five factors with the highest relative influence were included in the final model, such as the following model:

$$Δ CCT = A \cdot {I}_{1} + B \cdot {I}_{2} + C \cdot {I}_{3} + D \cdot {I}_{4} + E \cdot {r}_{b} + F ; \qquad ( 1 )$$

where ΔCCT is the change in central corneal thickness, I₁, I₂, I₃, and I₄ are parameters of isopach regularity (e.g., circularity and eccentricity) in the pachymetry map, r_{b} is a radius of the posterior corneal surface, and A, B, C, D, E, and F are coefficients. In an example implementation, the coefficients can have the following values: A=63.5, B=−245.7, C=246.9, D=−45.0, E=22.5, and F=44.9. It should be noted that the example predictive model in Eqn. (1) is independent of preoperative CCT.

When the predictive model(s) include one or more neural networks, training a neural network may include initializing the neural network, such as by computing, estimating, or otherwise selecting initial network parameters (e.g., weights, biases, or both). Training data can then be input to the initialized neural network, generating output as corneal improvement feature data. The quality of the corneal improvement feature data can then be evaluated, such as by passing the corneal improvement feature data to the loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. When the error has been minimized (e.g., by determining whether an error threshold or other stopping criterion has been satisfied), the current neural network and its associated network parameters represent the trained neural network.

The one or more trained predictive models are then stored for later use, as indicated at step 206. Storing the predictive model(s) may include storing model parameters (e.g., predictive input parameters, model coefficients, weights, biases, or combinations thereof), which have been computed or otherwise estimated by training the predictive model(s) on the training data. Storing the trained predictive model(s) may also include storing the particular predictive model structure to be implemented. For instance, data pertaining to the predictive model (e.g., number of predictive parameters to input, type of predictive parameters to input) may be stored.

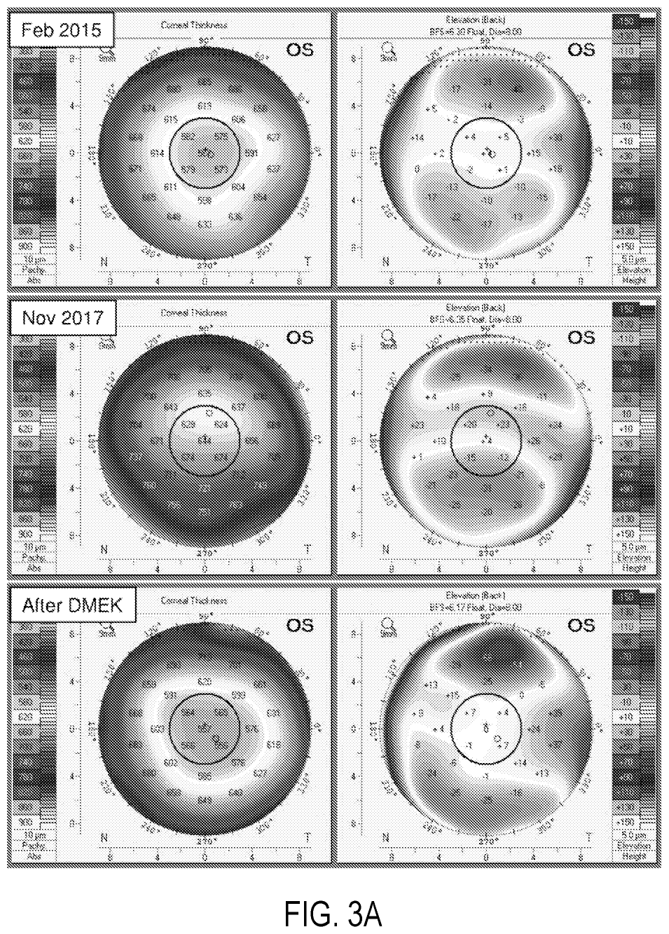

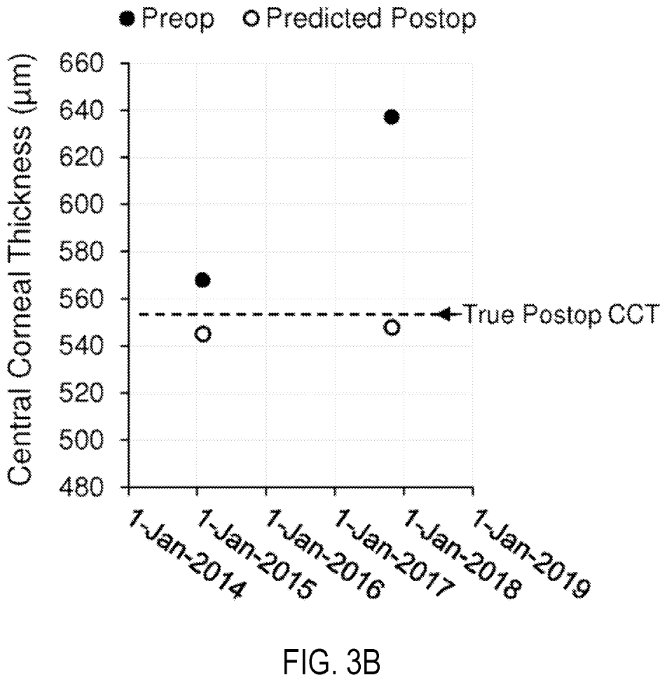

FIG. 3 illustrates one example implementation of predicting improvement in CCT from Scheimpflug tomography using the systems and methods described in the present disclosure. In February 2015, Scheimpflug tomography maps of the left cornea of a 61-year-old woman with Fuchs endothelial corneal dystrophy showed subtle loss of round and oval isopachs in the pachymetry map, possible early posterior surface depression inferior to the central cornea, and CCT of 568 mm. From those maps, the model predicted a 23-mm improvement in CCT with Descemet membrane endothelial keratoplasty (DMEK), which would have resulted in a postoperative CCT of 545 mm; no intervention was taken at the time because the patient was asymptomatic. By November 2017, the condition had progressed with obvious loss of circular isopachs, displacement of the thinnest point of the cornea, and central posterior depression; CCT had increased to 637 mm, and our model then predicted 89 mm of improvement, which would have resulted in CCT of 548 mm after DMEK. Observed CCT at steady state after DMEK in February 2018 was 557 mm. OS=left eye; Preop=before surgery; Postop=after surgery.

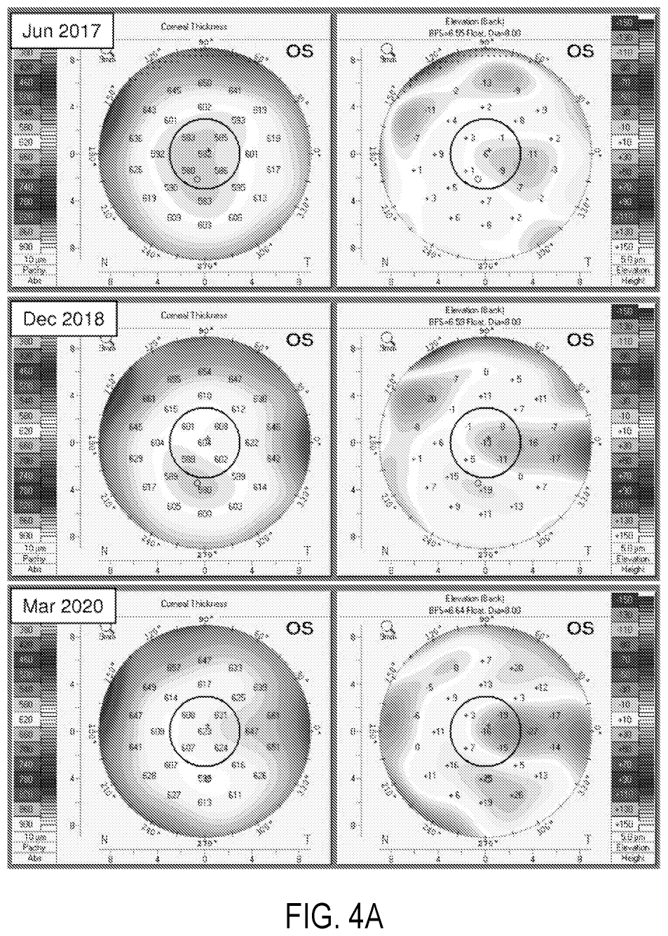

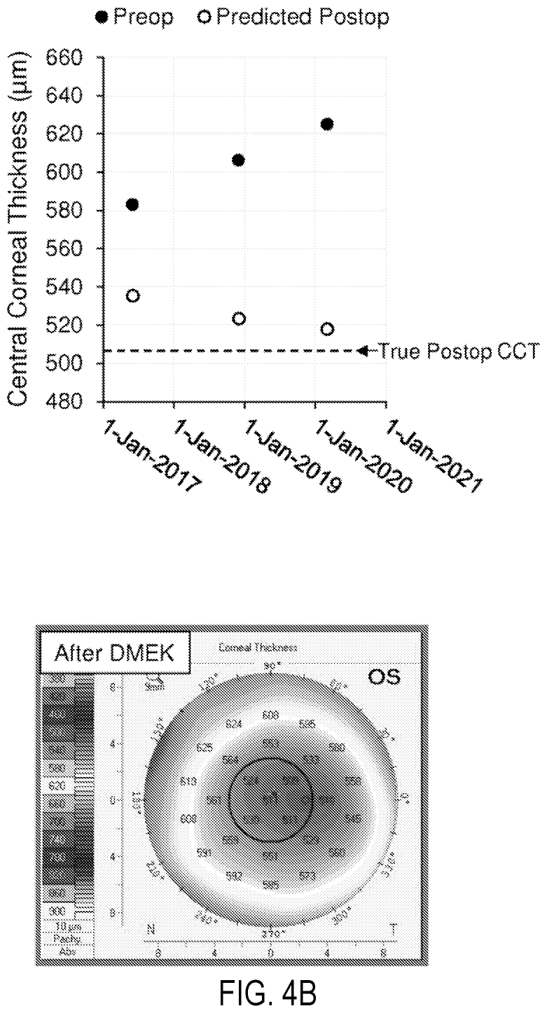

FIG. 4 illustrates another example of predicting improvement in CCT from Scheimpflug tomography using the systems and methods described in the present disclosure. In June 2017, Scheimpflug tomography maps of the left cornea of a 68-year-old woman showed irregular isopachs, displacement of the thinnest point, and focal posterior depression; although the patient had vision symptoms at that time, she deferred any intervention. Tomography images were repeated in December 2018 and March 2020 and showed worsening of the posterior elevation and pachymetry maps with a gradual increase in CCT. In June 2017, the model predicted 48 mm of improvement in CCT from 583 mm to 535 mm; in December 2018, the model predicted improvement of 83 mm, from 606 mm to 523 mm; in March 2020, the model predicted improvement of 107 mm, from 625 mm to 518 mm. Observed CCT at steady state after Descemet membrane endothelial keratoplasty (DMEK) in December 2020 was 509 mm. OS=left eye; Preop=before surgery; Postop=after surgery.

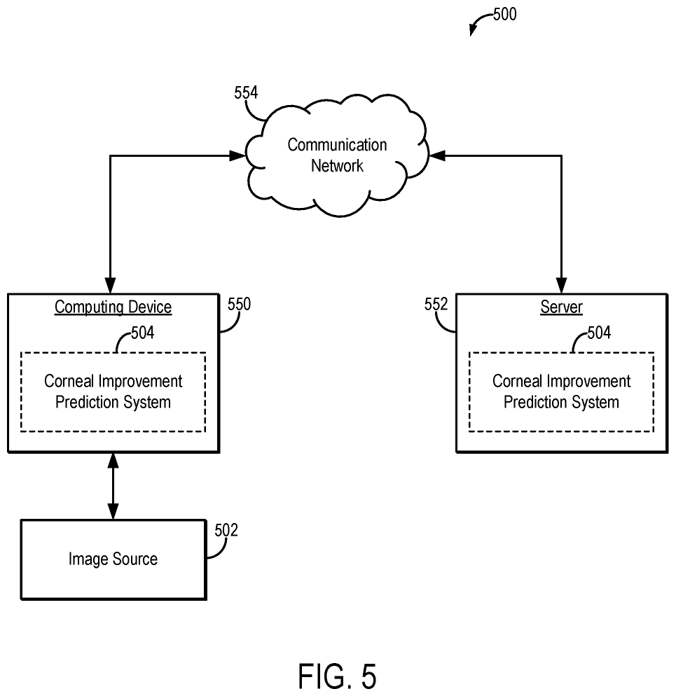

Referring now to FIG. 5, an example of a system 500 for predicting corneal improvement in accordance with some embodiments of the systems and methods described in the present disclosure is shown. As shown in FIG. 5, a computing device 550 can receive one or more types of data (e.g., Scheimpflug imaging data, tomography data, quantitative parameter data) from image source 502, which may be a Scheimpflug image source. In some embodiments, computing device 550 can execute at least a portion of a corneal improvement prediction system 504 to predict corneal improvement following therapy (e.g. DMEK) from data received from the image source 502.

Additionally or alternatively, in some embodiments, the computing device 550 can communicate information about data received from the image source 502 to a server 552 over a communication network 554, which can execute at least a portion of the corneal improvement prediction system. In such embodiments, the server 552 can return information to the computing device 550) (and/or any other suitable computing device) indicative of an output of the corneal improvement prediction system 504.

In some embodiments, computing device 550 and/or server 552 can be any suitable computing device or combination of devices, such as a desktop computer, a laptop computer, a smartphone, a tablet computer, a wearable computer, a server computer, a virtual machine being executed by a physical computing device, and so on. The computing device 550 and/or server 552 can also reconstruct images from the data.

In some embodiments, image source 502 can be any suitable source of image data (e.g., measurement data, images reconstructed from measurement data), such as a Scheimpflug imaging system, another computing device (e.g., a server storing image data), and so on. In some embodiments, image source 502 can be local to computing device 550. For example, image source 502 can be incorporated with computing device 550) (e.g., computing device 550 can be configured as part of a device for capturing, scanning, and/or storing images). As another example, image source 502 can be connected to computing device 550 by a cable, a direct wireless link, and so on. Additionally or alternatively, in some embodiments, image source 502 can be located locally and/or remotely from computing device 550, and can communicate data to computing device 550 (and/or server 552) via a communication network (e.g., communication network 554).

In some embodiments, communication network 554 can be any suitable communication network or combination of communication networks. For example, communication network 554 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 554 can be a local area network, a wide area network, a public network (e.g., the Internet), a private or semi-private network (e.g., a corporate or university intranet), any other suitable type of network, or any suitable combination of networks. Communications links shown in FIG. 5 can each be any suitable communications link or combination of communications links, such as wired links, fiber optic links, Wi-Fi links, Bluetooth links, cellular links, and so on.

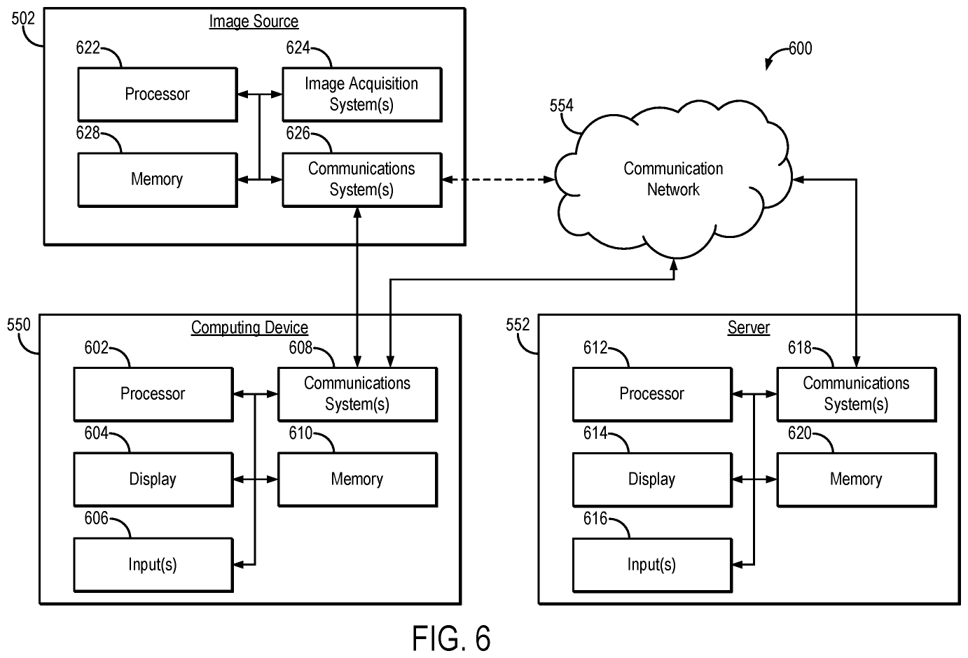

Referring now to FIG. 6, an example of hardware 600 that can be used to implement image source 502, computing device 550, and server 552 in accordance with some embodiments of the systems and methods described in the present disclosure is shown. As shown in FIG. 6, in some embodiments, computing device 550 can include a processor 602, a display 604, one or more inputs 606, one or more communication systems 608, and/or memory 610. In some embodiments, processor 602 can be any suitable hardware processor or combination of processors, such as a central processing unit (“CPU”), a graphics processing unit (“GPU”), and so on. In some embodiments, display 604 can include any suitable display devices, such as a computer monitor, a touchscreen, a television, and so on. In some embodiments, inputs 606 can include any suitable input devices and/or sensors that can be used to receive user input, such as a keyboard, a mouse, a touchscreen, a microphone, and so on.

In some embodiments, communications systems 608 can include any suitable hardware, firmware, and/or software for communicating information over communication network 554 and/or any other suitable communication networks. For example, communications systems 608 can include one or more transceivers, one or more communication chips and/or chip sets, and so on. In a more particular example, communications systems 608 can include hardware, firmware and/or software that can be used to establish a Wi-Fi connection, a Bluetooth connection, a cellular connection, an Ethernet connection, and so on.

In some embodiments, memory 610 can include any suitable storage device or devices that can be used to store instructions, values, data, or the like, that can be used, for example, by processor 602 to present content using display 604, to communicate with server 552 via communications system(s) 608, and so on. Memory 610 can include any suitable volatile memory, non-volatile memory, storage, or any suitable combination thereof. For example, memory 610 can include RAM, ROM, EEPROM, one or more flash drives, one or more hard disks, one or more solid state drives, one or more optical drives, and so on. In some embodiments, memory 610 can have encoded thereon, or otherwise stored therein, a computer program for controlling operation of computing device 550. In such embodiments, processor 602 can execute at least a portion of the computer program to present content (e.g., images, user interfaces, graphics, tables), receive content from server 552, transmit information to server 552, and so on.

In some embodiments, server 552 can include a processor 612, a display 614, one or more inputs 616, one or more communications systems 618, and/or memory 620. In some embodiments, processor 612 can be any suitable hardware processor or combination of processors, such as a CPU, a GPU, and so on. In some embodiments, display 614 can include any suitable display devices, such as a computer monitor, a touchscreen, a television, and so on. In some embodiments, inputs 616 can include any suitable input devices and/or sensors that can be used to receive user input, such as a keyboard, a mouse, a touchscreen, a microphone, and so on.

In some embodiments, communications systems 618 can include any suitable hardware, firmware, and/or software for communicating information over communication network 554 and/or any other suitable communication networks. For example, communications systems 618 can include one or more transceivers, one or more communication chips and/or chip sets, and so on. In a more particular example, communications systems 618 can include hardware, firmware and/or software that can be used to establish a Wi-Fi connection, a Bluetooth connection, a cellular connection, an Ethernet connection, and so on.

In some embodiments, memory 620 can include any suitable storage device or devices that can be used to store instructions, values, data, or the like, that can be used, for example, by processor 612 to present content using display 614, to communicate with one or more computing devices 550, and so on. Memory 620 can include any suitable volatile memory, non-volatile memory, storage, or any suitable combination thereof. For example, memory 620 can include RAM, ROM, EEPROM, one or more flash drives, one or more hard disks, one or more solid state drives, one or more optical drives, and so on. In some embodiments, memory 620 can have encoded thereon a server program for controlling operation of server 552. In such embodiments, processor 612 can execute at least a portion of the server program to transmit information and/or content (e.g., data, images, a user interface) to one or more computing devices 550, receive information and/or content from one or more computing devices 550, receive instructions from one or more devices (e.g., a personal computer, a laptop computer, a tablet computer, a smartphone), and so on.

In some embodiments, image source 502 can include a processor 622, one or more image acquisition systems 624, one or more communications systems 626, and/or memory 628. In some embodiments, processor 622 can be any suitable hardware processor or combination of processors, such as a CPU, a GPU, and so on. In some embodiments, the one or more image acquisition systems 624 are generally configured to acquire data, images, or both, and can include a Scheimpflug imaging system. Additionally or alternatively, in some embodiments, one or more image acquisition systems 624 can include any suitable hardware, firmware, and/or software for coupling to and/or controlling operations of a Scheimpflug imaging system. In some embodiments, one or more portions of the one or more image acquisition systems 624 can be removable and/or replaceable.

Note that, although not shown, image source 502 can include any suitable inputs and/or outputs. For example, image source 502 can include input devices and/or sensors that can be used to receive user input, such as a keyboard, a mouse, a touchscreen, a microphone, a trackpad, a trackball, and so on. As another example, image source 502 can include any suitable display devices, such as a computer monitor, a touchscreen, a television, etc., one or more speakers, and so on.

In some embodiments, communications systems 626 can include any suitable hardware, firmware, and/or software for communicating information to computing device 550 (and, in some embodiments, over communication network 554 and/or any other suitable communication networks). For example, communications systems 626 can include one or more transceivers, one or more communication chips and/or chip sets, and so on. In a more particular example, communications systems 626 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 628 can include any suitable storage device or devices that can be used to store instructions, values, data, or the like, that can be used, for example, by processor 622 to control the one or more image acquisition systems 624, and/or receive data from the one or more image acquisition systems 624; to images from data; present content (e.g., images, a user interface) using a display; communicate with one or more computing devices 550; and so on. Memory 628 can include any suitable volatile memory, non-volatile memory, storage, or any suitable combination thereof. For example, memory 628 can include RAM, ROM, EEPROM, one or more flash drives, one or more hard disks, one or more solid state drives, one or more optical drives, and so on. In some embodiments, memory 628 can have encoded thereon, or otherwise stored therein, a program for controlling operation of image source 502. In such embodiments, processor 622 can execute at least a portion of the program to generate images, transmit information and/or content (e.g., data, images) to one or more computing devices 550, receive information and/or content from one or more computing devices 550, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A method for predicting corneal improvement using Scheimpflug imaging, the method comprising:
(a) accessing Scheimpflug imaging data with a computer system, wherein the Scheimpflug imaging data have been acquired from a subject using a Scheimpflug imaging system;
(b) accessing a predictive model with the computer system, wherein the predictive model has been constructed to predict corneal improvement following a therapy based on preoperative Scheimpflug imaging data;
(c) applying the Scheimpflug imaging data to the predictive model, generating output as corneal improvement feature data that indicate a predicted corneal improvement following the therapy; and
(d) presenting the corneal improvement feature data to a user.

2. The method of claim 1, wherein the corneal improvement feature data comprise at least one predicted value of change in central corneal thickness.

3. The method of claim 1, wherein the corneal improvement feature data comprise a corneal thickness map that depicts predicted spatial distribution of change in corneal thickness over a region of a cornea of the subject.

4. The method of claim 1, wherein the corneal improvement feature data comprise at least one of a classification, quantitative score, probability of improvement, or other parameter indicating predicted corneal improvement following a therapy.

5. The method of claim 1, wherein the predictive model has been constructed using ensemble learning.

6. The method of claim 5, wherein the predictive model is constructed using ensemble learning comprising a boosting algorithm.

7. The method of claim 6, wherein the boosting algorithm is a gradient boosting algorithm.

8. The method of claim 1, wherein the Scheimpflug imaging data comprises at least one of Scheimpflug images, Scheimpflug tomography maps, quantitative parameters computed from Scheimpflug images, and quantitative parameters computed from Scheimpflug tomography maps.

9. The method of claim 8, wherein the Scheimpflug imaging data comprises Scheimpflug tomography maps comprising at least one of posterior elevation maps and pachymetry maps.

10. The method of claim 8, wherein the Scheimpflug imaging data comprises quantitative parameters computed from Scheimpflug tomography maps comprising at least one of isopach regularity and asphericity computed from pachymetry maps.

11. The method of claim 8, wherein the Scheimpflug imaging data comprises quantitative parameters computed from Scheimpflug tomography maps comprising radius of a posterior corneal surface computed from a posterior elevation map.

12. The method of claim 1, wherein the Scheimpflug imaging data comprises quantitative parameters computed from at least one of Scheimpflug imaging and Scheimpflug tomography maps, the quantitative parameters comprising at least one of irregular isopachs, displacement of a thinnest point of the subject's cornea, and volume of posterior depression.

13. The method of claim 1, wherein the Scheimpflug imaging data comprises a plurality of parameters of isopach regularity computed from pachymetry maps and a radius of a posterior corneal surface of the subject, and wherein the corneal improvement feature data comprise at least one predicted value of change in central corneal thickness.

14. The method of claim 13, wherein the predictive model is constructed using ensemble learning comprising a boosting algorithm.

15. The method of claim 14, wherein the boosting algorithm is a gradient boosting algorithm.

16. A method for predicting corneal improvement using Scheimpflug imaging, the method comprising:
(a) accessing Scheimpflug imaging data with a computer system, wherein the Scheimpflug imaging data have been acquired from a subject using a Scheimpflug imaging system and comprise Scheimpflug imaging parameters that are independent of corneal thickness;
(b) accessing a trained machine learning model with the computer system, wherein the trained machine learning model has been trained to predict corneal improvement following a therapy based on preoperative Scheimpflug imaging data;
(c) applying the Scheimpflug imaging data to the trained machine learning model, generating output as corneal improvement feature data that indicate a predicted corneal improvement following the therapy; and
(d) presenting the corneal improvement feature data to a user.

17. The method of claim 16, wherein the trained machine learning model comprises a neural network.

18. The method of claim 17, wherein the Scheimpflug imaging data comprise at least one Scheimpflug tomography map.

19. The method of claim 18, wherein the corneal improvement feature data comprise at least one of a classification, quantitative score, probability of improvement, or other parameter indicating predicted corneal improvement following a therapy.

20. The method of claim 16, wherein the machine learning model comprises a predictive model that has been trained using ensemble learning.

21. The method of claim 20, wherein the predictive model has been trained using ensemble learning comprising a boosting algorithm.

22. The method of claim 21, wherein the boosting algorithm is a gradient boosting algorithm.

23. The method of claim 16, wherein the Scheimpflug imaging data consist of Scheimpflug imaging parameters that are independent of corneal thickness.

24. The method of claim 16, wherein the machine learning model excludes preoperative central corneal thickness as an input parameter.
